(19) United States
(12) Patent Application Publication
(10) Pub. No.: US 2004/0214222 A1
(43) Pub. Date: Oct. 28, 2004

(54) Gene targeting methods and vectors

(75) Inventors: Robert Marshall Burgess JR., San Diego, CA (US); Henry Hongjun Ji, San Diego, CA (US)

Correspondence Address: Lisa A. Haile, J.D., Ph.D., GRAY CARY WARE & FREIDENRICH LLP, Suite 1100, 4365 Executive Drive, San Diego, CA, 92121-2133, US

(73) Assignee: GENOME BIOSCIENCES, LLC

(21) Appl. No.: 10/850,293

(22) Filed: May 19, 2004

Related U.S. Application Data
(62) Division of application No. 10/185,980, filed on Jun. 26, 2002.
(60) Provisional application No. 60/300,953, filed on Jun. 26, 2001.

(51) Int. Cl. C12Q 1/68; C12N 15/85

(52) U.S. Cl. 435/6; 435/455

(57) ABSTRACT

Methods and vectors are provided for the specific alteration of particular genetic loci in eukaryotic cells. One method includes the utilization of positive-positive selection (PPS) DNA vectors for the purpose of creating and identifying cells which have vector sequences integrated into host cell genome via site-specific homologous recombination. The method also comprises the utilization of sequences encoding in vivo detectable markers for the identification of cells which have exogenous vector sequences integrated into the genome of the host cell, either via site-specific homologous recombination or nonhomologous recombination or insertion. The invention also includes vectors for creating modifications in eukaryotic cells.

### RELATED APPLICATION DATA

The present application claims priority under 35 U.S.C. § 119(e) to U.S. Provisional Application Serial No. 60/300,953 filed on Jun. 26, 2001, which is incorporated by reference herein.

### FIELD OF THE INVENTION

The present invention relates generally to the manipulation of cells for the purposes of modifying genetic loci. More specifically, the invention relates to vectors and methods for generating genetic modifications in cells.

### BACKGROUND

Stable introduction of foreign genetic material into the genomes of both prokaryotic and eukaryotic organisms has been successfully accomplished in a variety of instances for various purposes such as the expression of an exogenous gene or the disruption of an endogenous locus. It is accomplished primarily through either random genomic insertion or site-specific homologous recombination. Random integration involves the insertion of a linearized DNA fragment into the genome of the host cell at locations which are, for the most part, non-site-specific. These insertions tend to exist as multimers or concatemers and most often do not result in the disruption and inactivation of a particular locus. The possibility also exists that endogenous loci may be disrupted by the random insertion event, thus often making analysis of the exogenous gene's effects on the cell or organism derived from the transformed cell difficult. In addition, a significant range of exogenous promoter activity may be observed depending upon the region of integration.

Insertion of DNA into the host genome via site-specific homologous recombination allows for the targeting of particular regions of the host genome for single copy integration of the exogenous DNA. Homologous recombination involves the exchange of significantly similar nucleotide sequences through the function of specific recombinase enzymes. Early experiments designed to manipulate cellular endogenous genomic DNA sequences with exogenous DNA in a site-specific manner focused on yeast as a model system. Recombination was demonstrated between the yeast genome and an exogenous plasmid introduced via transformation at the leu2.sup locus (Hinnen et al. (1978), Proc. Natl. Acad. Sci. U.S.A., 75, 1929) More recently the utilization of mammalian cellular homologous recombination capacities has allowed for the generation of specific mutated DNA sequences within the cellular endogenous genomic DNA. Both gain-of-function and loss-of-function alleles have been generated in stem cells and animals generated from said cells (see below). In addition, the application of positive-negative selection vectors and methods has accelerated the generation and study of cells and animals containing mutated DNA sequences (Capecchi et al. (1997) U.S. Pat. No. 5,631,153). To date, primarily two types of vectors have been designed which allow for targeting of a specific region of the genome for replacement of endogenous with exogenous DNA sequences. These vectors have proven to be sufficient for the generation of a variety of targeted alleles in a number of different cell types.

Insertion vectors contain two regions of homology flanking an internal nucleotide sequence encoding a selectable marker. The vector is linearized within one of the regions of homology. A single crossover event and homologous recombination results in a partial duplication of genomic sequences. Intrachromosomal recombination often results in exclusion of the endogenous duplicated sequences. A disadvantage to this type of targeting vector is the lack of a negative selectable marker which would allow for significant enrichment for correctly targeted events through elimination of cells which contain backbone or vector sequences. In addition, linearization within a region of homology reduces the amount of DNA sequence available for homologous recombination thus reducing the opportunity for strand exchange (Thomas et al. (1986), Cell, 44, 49). Finally, intrachromosomal recombination must occur within a defined region or regeneration of the wild-type organization of the locus may occur.

Replacement vectors contain two regions of homology usually flanking a positive selectable marker, such as the gene encoding neomycin phosphotransferase. A negative selectable maker is often located external but adjacent to one of the regions of homology to provide for enrichment of corrected targeted cell in the total population through elimination of cells containing the negative selectable cassette. Introduction of a replacement vector into cells followed by simultaneous or stepwise positive and negative selection results in the isolation of cells which have perhaps an eight to twelve-fold enriched probability of undergoing site-specific homologous recombination due to application of the negative selectable marker. In perhaps the first successful gene targeting experiments in mammalian cells, Capecchi et al. have demonstrated targeting of the mouse HPRT and int-2 loci via the use of replacement vectors (Capecchi et al., (1997), U.S. Pat. No. 5,631,153). A plethora of loci have since been successfully targeted, some by insertion vectors and the majority by replacement vectors. Many of these have included a negative selectable marker positioned external to either or both of the regions of homology, which often results in an increase in the efficiency of targeted allele identification. Yet a number of disadvantages exist with respect to the method and utility of replacement vectors and positive-negative selection. Utilization of a number of negative selectable cassettes such as HSV thymidine kinase requires the addition of an antibiotic or selective agent, gancyclovir for example, which may cause undo stress to the cells and unwanted or premature differentiation. In addition, selection of cells for enrichment with a negative selectable marker takes considerable time to allow for the cells to recover which have resistance to the drug due to absence of the selectable marker. As well, the enrichment factors typically obtained by this methodology are at most between eight to twelve-fold. As well, the creation of positive-negative selection vectors is often strategically difficult and time-consuming. The technology described in the present invention circumvents each of these issues.

A number of animals have also been created from embryonic stem cells which have particular loci mutated through site-specific homologous recombination. These include mice which are derived from chimeras produced by injection of blastocysts with embryonic stem cells targeted through homologous recombination at particular loci. Some examples include the p53 and paraxis loci (Donehower et al. (1992), Nature, 356, 215; Burgess et al. (1996), Nature, 384, 570). Pigs have also been derived from embryonic stem cells modified by homologous recombination include pigs (Butler et al. (2002), Nature, 415, 103).

### SUMMARY OF THE INVENTION

Methods are provided for the modification of genomic DNA sequences through homologous recombination of vector DNA with target DNA in eukaryotic cells. The first method entails first the transformation of a cell capable of undergoing homologous recombination with a vector, referred to herein as a positive-positive selection vector (PPS) containing sequences substantially similar to sequences present within the genome of the cell (FIG. 3). The majority of the vector integration into the genome of the host cell will occur in an essentially random manner, with no preference for particular regions of the genome. It is reasonably suggested, however, that a certain percentage of the PPS vector will integrate into the genome of the host cell via site-specific homologous recombination. Subsequent selection of the cells will allow for the isolation and identification of cells which have successfully undergone site-specific homologous recombination. The selection is based upon the organization and composition of the PPS replacement vector.

The vector is composed of a first DNA sequence which is significantly homologous to a sequence present within the host cell genome. In addition, the vector includes a third DNA sequence which is significantly homologous to other sequences within the host cell genome downstream or upstream of the first sequence. The vector contains between these two regions a second DNA sequence which is not significantly homologous to sequences present in the host genome and confers the ability to identify cells which have vector sequences integrated into said genome. A fourth DNA sequence is present within the vector positioned either 5′or 3′ to the first or second sequences which is not significantly homologous to sequences present in the host genome and confers a separate unique method to identify cells which have these sequences integrated into said genome. It is the utilization of the combination of the second and fourth DNA sequences that allows for the identification of cells which have undergone homologous recombination of the vector with endogenous sequences. A second procedure comprises the simultaneous or sequential cotransfection of vectors containing separate selectable markers for the purpose of creating and identifying cells which have vector sequences integrated into the host cell genome via site-specific homologous recombination. In addition, the invention includes cells and organisms generated from cells with specific genetic alterations through the implementation and use of provided procedures and vectors.

In a first embodiment, the invention provides a method for identifying a transformed cell which has undergone site-specific homologous recombination utilizing a PPS vector. The method includes

a) transforming cells with a PPS vector designed to undergo site-specific homologous recombination wherein the vector includes:

a first DNA sequence which is substantially homologous to an endogenous genomic sequence present within the host genome;

a second DNA sequence which encodes a positive selection characteristic in said cells and is non-homologous to cellular endogenous genomic sequences and therefore incapable of undergoing site-specific homologous recombination;

a third DNA sequence which is substantially homologous to an endogenous genomic sequence present within the host genome and is different from the first DNA sequence; and

a fourth DNA sequence which encodes a positive selection characteristic in said cells and is non-homologous to a cellular endogenous genomic sequence and therefore incapable of undergoing site-specific homologous recombination;

wherein the vector is capable of undergoing site-specific homologous recombination in cells through strand exchange between the first DNA sequence with endogenous target sequences and the third DNA sequence with endogenous target DNA sequences;

wherein the organization of the DNA sequences in the PPS vector is: the first DNA sequence which is substantially homologous to target DNA sequences, the second DNA second which encodes a positive selectable marker, the third DNA sequence which is substantially homologous to target DNA sequences, the fourth DNA sequence which encodes a positive selectable marker;

b) propagating cells to select for or enrich for those which have been successfully transformed with said PPS vector by selecting for the presence of the positive selectable marker gene product of said second DNA sequence, and

c) separating cells which have said second DNA sequence encoding a positive selectable marker from cells which have said fourth DNA sequence encoding a positive selectable marker. The method may further include d) characterizing the genomic DNA of said cells carrying the second DNA sequence encoding a positive selectable marker but not carrying the fourth DNA sequence encoding a positive selectable marker for the site-specific homologous recombination events which allow for modification of the cellular target DNA.

An object of the present invention is to provide site-specific homologous recombination methods for the targeting of specific regions of eukaryotic genomes for the purposes of modifying endogenous nucleotide sequences.

It is another object of the present invention to provide novel methods for the selection and detection of cells which have undergone site-specific homologous recombination.

It is a further object of the present invention to provide novel vectors for the application of the described methods.

It is still a further object of the present invention to provide cells which have been modified by site-specific homologous recombination methods described.

It is yet another embodiment of the present invention to provide transgenic animals and plants which have been modified by the site-specific homologous recombination and detection methods described.

### BRIEF DESCRIPTION OF FIGURES

FIG. 1 is a diagrammatic illustration of PPS targeting of the ptch2 locus.

FIG. 2 is a diagrammatic illustration of cotransformation targeting of the paraxis locus.

FIG. 3 is a flowchart representation of the strategy for the generation and identification of targeted cells utilizing PPS vectors and methods described herein.

FIGS. 4A and 4B are examples of the cell sorting density plots obtained upon the utilization of GFP and CFP, respectively.

FIGS. 5A and 5B illustrate a theoretical locus and corresponding PPS vectors which may be utilized to target said locus.

FIGS. 6A and 6B illustrate the two types of possible integration observed upon cotransformation of cells with DNA vectors.

### DETAILED DESCRIPTION OF THE INVENTION

The methods and vectors described in the present invention are utilized for the purpose of introducing modifications into cellular endogenous genomic target DNA sequences via site-specific homologous recombination.

The term “cellular endogenous genomic DNA sequence” is defined herein as nucleotide sequences present within the cellular genome which are capable of undergoing site-specific homologous recombination and may be utilized as a target for modification by the PPS vectors described herein. Sequences included within this definition may represent any coding or noncoding regions of specific genes present within the cellular genome. Genes encoding such protein products as structural proteins, secreted proteins, hormones, receptors, enzymes, transcription factors are included in this definition. These sequences may also represent regulatory element indentity such as promoters, enhancers or repressor elements. The organization of the cellular endogenous genomic target DNA sequence is generally similar to specific sequences present within the PPS vector. That is, it contains sequences which are substantially homologous to sequences present within the PPS vector that allow for site-specific homologous recombination to occur.

The vector is composed of a first DNA sequence which is significantly homologous to a sequence present within the host cell genome. In addition, the vector includes a third DNA sequence which is significantly homologous to other sequences within the host cell genome downstream or upstream of the first sequence. The vector contains between these two regions a second DNA sequence which is not significantly homologous to sequences present in the host genome and confers the ability to identify cells which have vector sequences integrated into said genome. A fourth DNA sequence is present within the vector positioned either 5′or 3′ to the first or second sequences which is not significantly homologous to sequences present in the host genome and confers a separate unique method to identify cells which have these sequences integrated into said genome. It is the utilization of the combination of the second and fourth DNA sequences that allows for the identification of cells which have undergone homologous recombination of the vector with endogenous sequences. A second procedure comprises the simultaneous or sequential cotransfection of vectors containing separate selectable markers for the purpose of creating and identifying cells which have vector sequences integrated into the host cell genome via site-specific homologous recombination. In addition, the invention includes cells and organisms generated from cells with specific genetic alterations through the implementation and use of provided procedures and vectors.

The term “site-specific homologous recombination” refers to strand exchange crossover events between DNA sequences substantially similar in nucleotide composition. These crossover events may take place between sequences contained in the PPS vector and cellular endogenous genomic DNA sequences. In addition, it is possible that more than one site-specific homologous recombination event may occur between DNA sequences present in the PPS vector and cellular endogenous genomic sequences which would result in a replacement event in which DNA sequences contained within the PPS vector have replaced specific sequences present within the cellular endogenous genomic sequences. As well, a single site-specific homologous recombination event may occur between DNA sequences present in the PPS vector and cellular endogenous genomic sequences which would result in an insertion event in which the majority or the entire PPS vector is inserted at a specific location within the cellular endogenous genomic sequences.

The term “cotransformation” refers to the process of either sequentially or simultaneously introducing exogenous DNA sequences into living cells for the purposes of mutating cellular endogenous genomic DNA sequences via site-specific homologous recombination.

The term “first DNA sequence” refers to DNA sequences present within the PPS vector which are substantially homologous to cellular endogenous genomic sequences. It is these sequences which are predicted to undergo site-specific homologous recombination upon their introduction into cells capable of undergoing said recombination which contain similar sequences.

The term “second DNA sequence” refers to sequences encoding a positive selectable marker which may or may not be expressed independently of cellular target sequences due to the presence of absence of a promoter and regulatory elements upstream of the positive selectable marker. The positive selectable marker is positioned between the first and third DNA sequences which are substantially homologous to cellular endogenous genomic DNA sequences. The positive selectable marker is nonhomologous to cellular endogenous genomic DNA sequences and therefore incapable of site-specific homologous recombination with this sequences.

The term “third DNA sequence” refers to DNA sequences present within the PPS vector which are substantially homologous to cellular endogenous genomic sequences yet are different but possibly adjacent or within reasonable proximity to those of the first DNA sequence. It is these sequences which are predicted to undergo site-specific homologous recombination upon their introduction into cells capable of undergoing said recombination which contain similar sequences.

The term “fourth DNA sequence” refers to a positive selectable marker positioned external to the first and third DNA sequences. The positive selectable marker encoded by the fourth DNA sequence contains its own promoter and regulatory elements and therefore its expression is independent of regulatory elements present within the cellular endogenous genomic target DNA sequences. The positive selectable marker is nonhomologous to cellular endogenous genomic DNA sequences and therefore incapable of site-specific homologous recombination with this sequences.

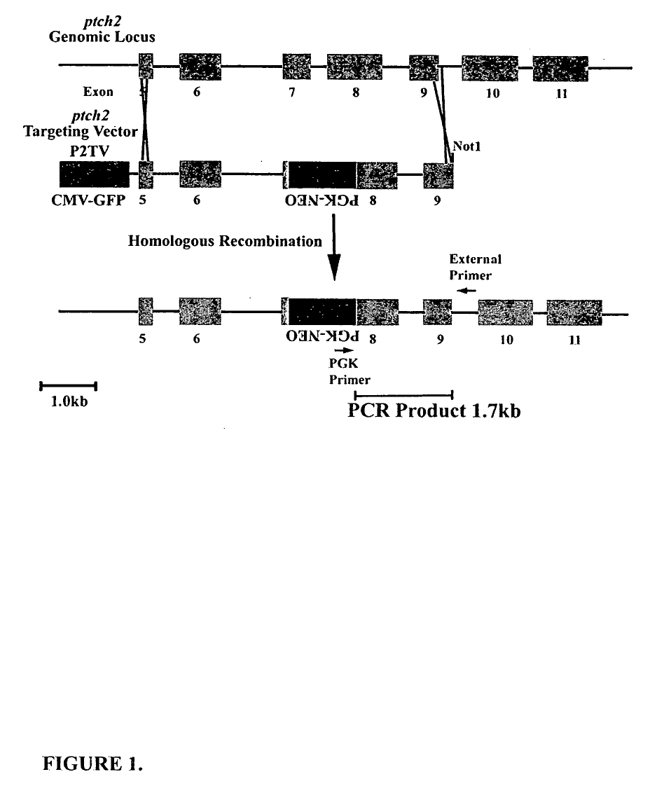

In a replacement PPS vector, the first, second, third and fourth DNA sequences are organized such that the second DNA sequence, which encodes a positive selectable marker, is positioned between the first and third DNA sequences and the fourth DNA sequence is place either 5′ or 3′ of the first or third DNA sequences. FIG. 1 illustrates the organization of two PPS vectors utilized for site-specific homologous recombination.

The external positive selectable marker encoded by the fourth DNA sequence may be positioned either upstream or downstream of the first and third sequences contained in the PPS vector. Upstream generally refers to 5′ and downstream generally refers to 3′ of the first and third DNA sequences in a vector which has both the first and third DNA sequences in an orientation similar to that of cellular endogenous genomic sequences. It is to be clarified that 5′ and 3′ refer to the first and third DNA sequences respectively. This organization represents a replacement vector. It is possible that the second DNA sequence, which encodes the internal positive selectable marker, may be inverted with respect to the first and third DNA sequences and still retain expressability and functionality. In addition, it is possible that portions of the first and third sequences in the PPS vector are inverted with respect to one another in comparison to similar sequences in the cellular target DNA. This type of organization represents an insertion vector. Insertion vector generally incorporate the majority of the vector sequence into the cellular genome upon site-specific homologous recombination.

An additional external positive selectable marker may be encoded, such as by a fifth DNA sequence, which is placed in a position opposite that of the fourth DNA sequence encoding a separate positive selectable marker. The term “opposite” refers to a position external to either the first or third DNA sequences and located on the other side of these sequences, i.e. either 5′ or 3′ in relation to the positive marker encoded by the fourth DNA sequence.

In an insertion PPS vector, the first, second and third sequences are organized such that the third sequence has an inverted 5′ to 3′ orientation with respect to the first sequence upon linearization of the vector. Said inverted orientation allows for the insertion of the vector at a site-specific location upon site-specific homologous recombination between the PPS vector and cellular endogenous genomic DNA sequences. In the majority of the cases the entire vector will be inserted and portions of the substantially homologous DNA sequences duplicated. The fourth DNA sequence encoding a positive selectable marker may or may not be included in the PPS vector

The length of the PPS vector will vary depending upon the choice of positive selectable markers, the presence or absence of promoters capable of driving the expression of the positive selectable marker encoded by the second DNA sequence, the length of the first and third DNA sequences required for appropriate homologous recombination, the size of the base vector and the choices for selection of the plasmid vector in bacteria such as ampicillin resistance and the size of the origin of replication for the plasmid backbone. It is reasonably estimated, however, based upon the sizes of known plasmids and positive selectable markers, that the entire vector will be at least several kilo basepairs in length.

The term “functional” is defined herein with respect to positive selectable markers as conferring the ability of markers to allow for the detection and isolation of cells containing DNA encoding the positive selectable marker and to allow for the differentiation of these cells from cells which contain either no positive selectable marker or a positive selectable marker which is unique in comparison to the first positive selectable marker. A number of selective agents may be utilized for the detection of positive selectable marker presence within cells. These include, but are not limited to, G418, hypoxanthine, bleomycin, hygromycin, puromycin and blasticidin, for example and are listed in Table I. In addition, positive selectable markers which do not require the addition of agents for the identification of marker presence are considered functional if they allow for the isolation of cells containing said selectable marker from cells which contain different selectable markers or no selectable marker. Some examples include, but are not limited to, the fluorescent proteins GFP, CFP, YFP, RFP, dsRED and HcRED, also listed in Table I.

TABLE I
Positive Selectable Markers Utilized in PPS Vectors

| Positive Marker | Selection Agent | Method for Detection |
|---|---|---|
| NeoR | Kanamycin | Cell Survival |
| NeoR | G418 | Cell Survival |
| HygroR | Hygromycin | Cell Survival |
| hisDR | Histidinol | Cell Survival |
| GPT | Xanthine | Cell Survival |
| BleoR | Bleomycin | Cell Survival |
| HPRT | Hypoxanthine | Cell Survival |
| GFP | UV Light | Fluorescence |
| CFP | UV Light | Fluorescence |
| YFP | UV Light | Fluorescence |
| RFP | UV Light | Fluorescence |
| dsRED | UV Light | Fluorescence |
| HcRED | UV Light | Fluorescence |

The PPS vector includes two regions of homology, DNA sequences one and three, which are substantially homologous to regions of the host genome. Typically, the vector has lengths of homology for the first and third DNA sequences which are between about 50 base pairs and 50,000 base pairs. It also includes DNA sequences two and four, which encode two positive selectable markers that allow for the identification of the presence or absence of the PPS vector integrant and portions thereof within the host genome. The second DNA sequence encodes a positive selectable marker, such as, but not limited to, cyan fluorescent protein (CFP) for example, and is positioned between the two regions of homology, thus it will be included in the host genome integrant should site-specific homologous recombination occur. The fourth DNA sequence encodes another positive selection marker, such as, but not limited to, green fluorescent protein (GFP) for example, and is positioned outside of the regions of homology and thus will not be incorporated into the host genome upon homologous recombination. The selection process involves sorting of cells either under a microscope or through a FACS cell sorting apparatus which will allow for the simultaneous and separate isolation of cells which contain the second DNA sequences encoding a positive selection marker from cells containing the fourth DNA sequences including the second positive selectable marker. Cells may subsequently be propagated in tissue culture and genotyped for correct site-specific homologous recombination gene targeting events. The utilization of positive selectable markers for the isolation of cells which have undergone site-specific homologous recombination allows for a substantial improvement over existing methodologies for gene targeting.

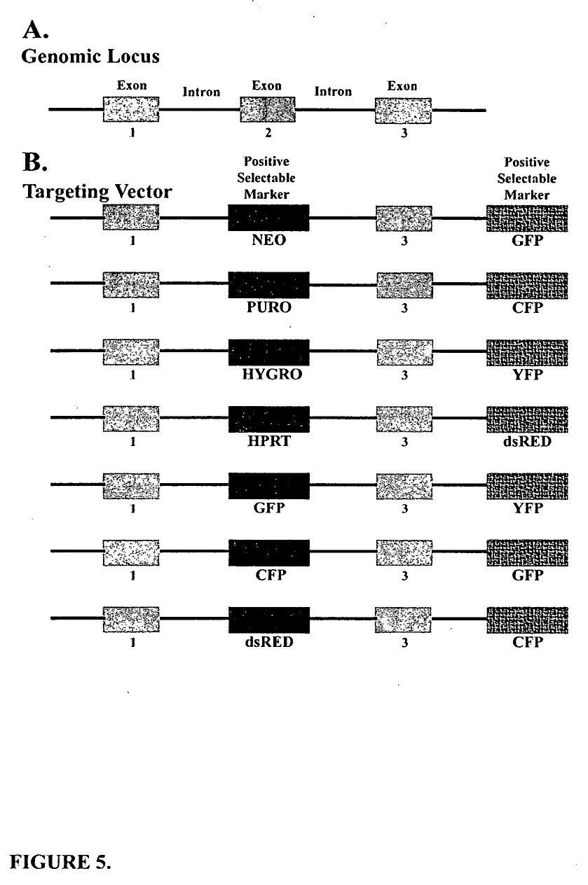

The PPS vectors utilized in the first method of the presently described invention are organized such that the second DNA sequence which encodes one positive selectable marker is operatively positioned between the two regions of homology and the fourth DNA sequence which encodes another positive selectable marker is operatively positioned externally or outside of the two regions of homology. It is possible that the second DNA sequence may be positioned in such a fashion as to disrupt or replace exonic or coding sequences of the endogenous region of the genome at which site-specific homologous recombination may occur thus rendering the endogenous locus inactive and thus nonfunctional (FIG. 5).

In one aspect, the second DNA sequence may be positioned such that it replaces or inserts into regions of the genome which do not confer exonic or coding sequences such as introns, untranslated regions of exons or regulatory element regions such as promoters. In this scenario it may be possible to select for cells which have undergone site-specific homologous recombination at the locus without inactivating that particular locus.

In another aspect, the second DNA sequence may also include regulatory elements unique to that sequence and may be positioned in such a manner that it introduces novel regulatory elements within the region of the genome selected for site-specific recombination. The invention includes the PPS vectors described for the purposes of performing site-specific homologous recombination and subsequent identification of cells which have undergone said recombination.

The presently described invention also includes cells which have undergone site-specific homologous recombination in accordance with the PPS vectors and methods for identification described herein.

In addition, the presently described invention includes transgenic non-human animals which have been derived from cells which have undergone site-specific homologous recombination utilizing PPS vectors and methods described herein.

Also included are transgenic plants which have been derived from cells which have undergone site-specific homologous recombination utilizing PPS vectors and methods described herein. Plants have previously been demonstrated to undergo site-specific homologous recombination as well as gene targeting via positive-negative selection and are therefore amenable to the PPS vectors and methods described herein (Siebert et al. (2002), Plant Cell, 14, 1121; Hanin et al. (2001), Plant J., 28, 671; Xiaohui et al. (2001), Gene, 272, 249).

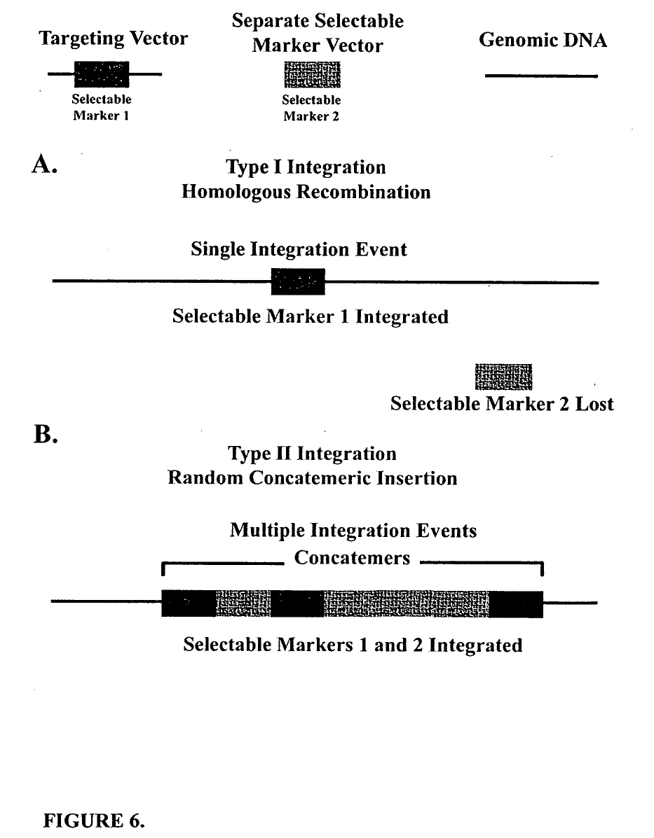

The second method, herein referred to as the “cotransformation” method, involves transformation of a cell capable of undergoing homologous recombination with a first DNA vector containing sequences substantially similar to sequences present within the genome of the cell followed by or simultaneously with a second DNA vector (FIG. 6). The majority of the vector(s) integration into the genome of the host cell will occur in an essentially random manner, with no preference for particular regions of the genome. It is reasonably suggested, however, that a certain percentage of the first DNA vector will integrate into the genome of the host cell via site-specific homologous recombination. Subsequent selection of the cells will allow for the isolation and identification of cells which have successfully undergone site-specific homologous recombination. The selection is based upon the organization and composition of the first DNA vector and the selectable marker identity in the second DNA vector. The first DNA vector includes two regions of homology, DNA sequences one and three, which are substantially homologous to regions of the host genome. It also includes DNA sequences two and an optional DNA sequence four, which encode two positive selectable markers that allow for the identification of the presence or absence of the PPS vector integrant and portions thereof within the host genome. The second DNA sequence encodes a positive selectable marker, such as, but not limited to, neomycin phosphotransferase for example, and is positioned between the two regions of homology, thus it will be included in the host genome integrant should site-specific homologous recombination occur. The selection process involves sorting of cells either under a microscope or through a FACS cell sorting apparatus which will allow for the simultaneous and separate isolation of cells which contain DNA sequences encoding the positive selection marker of the first DNA vector from cells containing DNA encoding selectable marker of the second DNA vector. The selection process may alternatively or in conjunction with the first selection process involve the utilization of a negative selectable marker (see Table II) in the second DNA vector which allows for the killing of cells containing said DNA vector. Cells may subsequently be propagated in tissue culture and genotyped for correct site-specific homologous recombination gene targeting events. The utilization of selectable marker cotransformation for the isolation of cells which have undergone site-specific homologous recombination allows for a substantial improvement over existing methodologies for gene targeting.

The first DNA vector utilized in the cotransformation method of the presently described invention are organized such that the second DNA sequence which encodes one positive selectable marker is operatively positioned between the two regions of homology (see Table I). It is possible that the second DNA sequence may be positioned in such a fashion as to disrupt or replace exonic or coding sequences of the endogenous region of the genome at which site-specific homologous recombination may occur thus rendering the endogenous locus inactive and thus nonfunctional.

The second DNA sequence may be positioned such that it replaces or inserts into regions of the genome which do not confer exonic or coding sequences such as introns, untranslated regions of exons or regulatory element regions such as promoters. In this scenario it may be possible to select for cells which have undergone site-specific homologous recombination at the locus without inactivating that particular locus.

In a third scenario, it is possible that the second DNA sequence may also include regulatory elements unique to that sequence and may be positioned in such a manner that it introduces novel regulatory elements within the region of the genome selected for site-specific recombination.

The sequence composition of the second and fourth DNA sequences which encode positive selectable markers are generally nonhomologous to cellular endogenous genomic DNA sequences and therefore are not capable of undergoing site-specific homologous recombination. Thus all site-specific homologous recombination is the result of the first and third DNA sequences which encode regions that are substantially homologous to cellular endogenous genomic DNA sequences and therefore capable of undergoing the strand exchange crossover process. In addition, the second and fourth DNA sequences which encode the positive selectable markers may be positioned in an orientation-independent manner with respect to each other and with respect to the cellular endogenous genomic DNA sequences. Such a positioning for the second DNA sequence, however, requires expression of the positive selectable marker which is independent of cellular endogenous genomic DNA regulatory elements.

A number of preferred positive selectable markers exist for the second DNA sequence (see Table I). These sequences allow for selection of cells carrying the positive selectable marker in order to distinguish said cells from those which do not carry the positive selectable marker. Perhaps the most widely utilized positive selectable marker utilized as the second DNA sequence encodes the neomycin phosphotransferase gene product. Other positive selectable markers appropriate for the second DNA sequence include, but are not limited to, those which code for blasticidin resistance, puromycin resistance, bleomycin resistance and hygromycin resistance (FIG. 5). Several of these positive selectable markers may also be applied to the use of PPS vectors for site-specific homologous recombination in plants. In addition, these markers may be applied to the use of cotransformation methods.

The term “negative selectable marker” includes any particular gene, DNA sequence, protein, peptide or amino acid sequences which, when introduced into cells or within the proximity of cells, confers the ability to eliminate cells from a general population through the act of cell killing.

The term “negative selection” refers to the act of selecting against cells through the implementation of methodologies which allow for the killing of said cells.

With respect to the cotransformation methodology described herein, a number of negative selectable markers may be utilized to enhance or enrich for the possibility of identifying a cell which has undergone site-specific homologous recombination. These include, but are not limited to, thyrnidine kinase, diphtheria toxin A chain, hprt and gpt and see Table II.

TABLE II
Negative Selectable Markers Utilized in Cotransformation
Methodologies

| | | Method |
|---|---|---|
| Negative Marker | Selection Agent | for Detection |
| Diphtheria Toxin | None | Cell Killing |
| Ricin Toxin | None | Cell Killing |
| HPRT | 6-Thioguanine | Cell Killing |
| HSV-Thymidine Kinase | Gancyclovir, Acyclovir, | Cell Killing |
| | FIAU | |
| GPT | 6-Thioguanine | Cell Killing |
| Cytosine Deaminase | 5-Fluoro-Cytosine | Cell Killing |

A “mutating DNA sequence” is herein referred to as any sequence which changes the nucleotide composition of cellular endogenous genomic DNA sequences. Said change may result in an inactivation of the functional capacity of the cellular DNA sequence. Said change may also enhance the functional capacity of the cellular DNA sequence or it may have no effect on the functional capacity of the cellular DNA sequence.

A “mutated DNA sequence” is herein referred to as any cellular endogenous genomic DNA sequence which has undergone alteration through the utilization of PPS vectors. It is generally anticipated that mutated DNA sequences will be generated upon site-specific homologous recombination between the PPS vector and cellular endogenous genomic DNA sequences.

“Mutated target cells” are cells capable of undergoing site-specific homologous recombination which have a mutated DNA sequence established within the cellular genome through the application of mutating DNA sequences present in the PPS vectors described herein.

The term “substantially nonhomologous DNA” refers to DNA sequences which do not contain nucleotide sequences similar enough to target DNA sequences to allow for the process of site-specific homologous recombination to occur. Dissimilar sequences of this capacity fail to undergo site-specific homologous recombination with target DNA sequences due to the mismatch of base pair composition between the two sequences.

There are a number of applicable advantages to establishing mutated DNA sequences within a cellular genome. X-linked genes, for example, may be analyzed for functional relevance in tissue culture if the particular cell type targeted by the PPS vector is of male origin. In addition, manipulation of embryonic stem cells via PPS vectors may allow for the creation of animal models for the study of human disorders. The p53 locus, for example, has been successfully inactivated via positive-negative selection technology in mouse embryonic stem cells and those cells utilized for the creation of mice deficient in the protein product encoded by this locus (Donehower et al. (1992), Nature, 356, 215). These mice are developmentally normal but susceptible to spontaneous tumors. PPS vectors and technology allow for the generation of similar genetic modifications in embryonic stem cells and animals created from said cells. Other uses of PPS vectors and technology include the generation of gain-of-function alleles which may allow for the study of a variety of cellular and physiologic phenomena. Many proto-oncogenes have been analyzed as gain-of-function alleles including c-myc, cyclin D1 and ErbB-2 (for review see Hutchinson et al. (2000), Oncogene, 19, 130). Use of the PPS vectors and methods described herein efficiently allow for both loss- and gain-of-function studies in embryonic stem cells as well as transgenic animals derived from these cells.

PPS vectors and methods as well as cotransformation methods are utilized for the purposes of creating and identifying cells which have undergone site-specific homologous recombination between the vector and cellular endogenous genomic target sequences. The vectors substantially enrich for the identification of cells which have undergone said process. To “substantially enrich” refers to the ability to significantly increase the likelihood of identifying cells for which site-specific homologous recombination between the vector and cell DNA sequences. The significant increase in likelihood is at least two-fold of homologous recombination events when compared to nonspecific insertion or integration events, preferably at least 10-fold, more preferably at least 100-fold and even more preferably at least 10,000-fold. Substantially enriched cell populations derived from the use of PPS vectors include around 1%, more preferably 10%, and even more preferably 99% of cells isolated have undergone site-specific homologous recombination between PPS vector sequences and cellular endogenous genomic target sequences.

It is possible that PPS vectors or vectors of the cotransformation methodology may be designed to drive the expression of the positive selection marker under the control of regulatory elements endogenous to the particular gene targeted by the PPS vector. In such an instance, the vector is constructed such that the sequences encoding the positive selectable marker lack an upstream element sufficient to drive expression of that marker. Homologous recombination between the PPS vector and cellular endogenous genomic target sequence provides regulatory elements specific for the targeted gene which subsequently drive the expression of the positive selectable marker. The positive selectable marker will most often not be expressed unless site-specific homologous recombination occurs, thereby providing endogenous cellular regulatory elements sufficient to drive expression of the marker. An example of the organization of such a vector is to position the second DNA sequence encoding the positive selectable marker between the first DNA sequence which is substantially homologous to cellular endogenous genomic target DNA and contains a promoter and portion of a 5′ untranslated region, and the third DNA sequence which is substantially homologous to cellular endogenous genomic target DNA and contains a portion of an intron and a downstream exon. Site-specific homologous recombination between the PPS vector and cellular endogenous genomic target sequences results in the positioning of the positive selectable marker under the control of endogenous regulatory elements.

A variety of scenarios are possible for the positioning of the second DNA sequence encoding the positive selectable marker which may result in a number of phenotypes with respect to the function of the gene targeted for modification. It is possible, for example, to achieve site-specific homologous recombination between the PPS vector or the cotransformation vector and cellular endogenous genomic target sequences without disruption of endogenous loci. This is accomplished through the positioning of the second DNA encoding the positive selectable marker within an intron or noncoding region such that introduction of said positive selectable marker into the genome of the host cell does not disrupt regulatory, exonic or coding sequences. An example of the organization of such a vector is to position the second DNA sequence encoding the positive selectable marker between the first DNA sequence which is substantially homologous to cellular endogenous genomic target DNA and contains an exon and portion of an intron, and the third DNA sequence which is substantially homologous to cellular endogenous genomic target DNA and contains a portion of an intron and a downstream exon. Site-specific homologous recombination between the PPS vector and cellular endogenous genomic target sequences subsequently results in the positioning of the positive selectable marker within the intron and thus not disrupting critical exonic coding sequences. A requirement is that the positive selection marker must be under the control of regulatory elements present within the PPS vector.

The introduction of a mutating DNA sequence into the genome of target cells capable of undergoing site-specific homologous recombination is not restricted to the creation of a loss-of-function or gain-of-function allele. It is possible, for example, to introduce exogenous regulatory sequences for the purposes of driving expression of particular cellular endogenous loci targeted by site-specific homologous recombination to novel tissue- and/or cell-type-specific regions within cells or transgenic animals or plants created from cells targeted by PPS vectors or cotransformation methodologies. The use of PPS vectors and methodologies as well as cotransformation methodologies for this purpose allows for an ability to dictate or control the spatial and temporal expression pattern of virtually any gene which is capable of undergoing site-specific homologous recombination. An example of the application of the technology described herein for this purpose would be to introduce the promoter and regulatory elements from the Pit-1 locus upstream of sequences coding for the proto-oncogene c-myc. Such a scenario would allow for ectopic expression of c-myc in somatotrope, lactotrope and thyrotrope cells of the developing and adult pituitary and provide a model for pituitary tumorigenesis (Rhodes et al. (1996), Mol. Cell Endocr., 124, 163; Baxter et al. (2001), 75, 9790). Table III lists a number of characterized regulatory sequences which may be utilized to drive the expression of endogenous loci through PPS and cotransformation methodologies.

TABLE III
Regulatory Element Examples.

| Regulatory Region | Expression Pattern |
|---|---|
| Pit-1 | Pituitary |
| Prolactin | Pituitary Lactotropes |
| Growth Hormone | Pituitary Somatotropes |
| Myogenin | Skeletal Muscle |
| Alpha Crystallin | Lens of the eye |
| Protamine | Testes |
| P-lim | Rathke's Pouch, motor neurons |
| GATA-3 | Liver |
| Insulin | Pancreas |
| GnRH | Hypothalamus |
| Dystrophin | Cardiac and Skeletal Muscle |

It is understood that any cell type that is capable of undergoing site-specific homologous recombination may be manipulated by PPS vectors and methodology as well as by cotransformation methodology for the purposes of mutating cellular endogenous genomic DNA sequences. Cells capable of undergoing site-specific homologous recombination may be derived from a variety of organisms and species including, but not limited to, human, murine, ovine, porcine, bovine, simian, canine and feline. In general, any eukaryotic cell capable of undergoing site-specific homologous recombination may be targeted successfully for the generation of a mutated DNA sequence within the cellular endogenous genomic DNA by PPS vectors and methodology as well as by cotransformation methodology.

When the creation of a transgenic non-human animal containing modification produced through the utilization of PPS vectors and methodology or cotransformation methodology is desired, the preferred cell type is embryonic stem cells. These cells are generally derived from the inner cell mass of preimplantation embryos and propagated in tissue culture for genetic manipulation. Upon mutating the cellular endogenous genomic DNA sequences through the application of PPS vectors and methodology or cotransformation methodology, the cells are introduced into blastocysts via microinjection techniques and said blastocysts implanted into pseudopregnant female hosts (Hogan et al. (editor) (1994), Manipulating the Mouse Embryo, A laboratory manual, Cold Spring Harbor Laboratory Press, New York). Alternatively, morula aggregation methods may be implemented for the creation of embryos containing genetically modified stem cells (Kong et al. (2000), Lab Anim., 29, 25). Embryos which survive through postnatal stages often exhibit a chimeric cellular content in which a certain percentage of cells are derived from blastocyst origin and a certain percentage of cells are derived from those mutated by PPS vectors and technology or cotransformation methodology. Chimeric animals may subsequently be breed to hetero- and homozygosity for the allele mutated by PPS vectors and technology and cotransformation methodology.

The PPS and cotransformation vectors and methodology described herein may be utilized for the purposes of correcting specific genetic defects in humans. It is possible, for example, to generate a mutated DNA sequence in human stem cells through site-specific homologous recombination between a PPS vector and cellular endogenous genomic DNA sequences and subsequently transplant those cells into patients for the correction of a specific genetic disorder or supplementation of a particular gene product. A similar scenario may apply for cotransformation methodologies. Another potential use for gene inactivation is disruption of proteinaceous receptors on cell surfaces. For example cell lines or organisms wherein the expression of a putative viral receptor has been disrupted using an appropriate PPS vector can be assayed with virus to confirm that the receptor is, in fact, involved in viral infection. Further, appropriate PPS vectors may be used to produce transgenic animal models for specific genetic defects. For example, many gene defects have been characterized by the failure of specific genes to express functional gene product, e.g. .alpha. and .beta. thalassema, hemophilia, Gaucher's disease and defects affecting the production of .alpha.-1-antitrypsin, ADA, PNP, phenylketonurea, familial hypercholesterolemia and retinoblastemia. Transgenic animals containing disruption of one or both alleles associated with such disease states or modification to encode the specific gene defect can be used as models for therapy. For those animals which are viable at birth, experimental therapy can be applied. When, however, the gene defect affects survival, an appropriate generation (e.g. F0, F1) of transgenic animal may be used to study in vivo techniques for gene therapy.

PPS and cotransformation vectors are designed for the specific purposes of mutating DNA sequences in the endogenous genomic DNA of cells capable of undergoing site-specific homologous recombination. The components of the PPS vector include at least one region of DNA which is substantially homologous to cellular endogenous genomic DNA sequences, one DNA sequence encoding a positive selectable marker capable of conferring the ability to identify cells containing the positive selectable marker from cells which do not contain sequences encoding the positive selectable marker, at least one other DNA sequence element encoding a unique positive selectable marker which allows for the identification and separation of cells which contain sequences encoding this marker from cells which do not contain sequences encoding this marker (FIG. 5).

In addition, it is preferable that the PPS or cotransformation vector be linearized prior to its introduction into cells for the purposes of mutating cellular endogenous genomic DNA sequences as linear vectors exhibit significantly higher targeting frequencies than those circular (Thomas et al. (1986), Cell, 44, 49). It is, however, possible to successfully utilize PPS vectors for these purposes without linearization.

For the purposes of targeting different alleles, it may be necessary to utilize different regulatory elements for the expression of the positive selectable markers, specifically the marker encoded by the second DNA sequence which is positioned between the two regions of substantial homology in a replacement vector. By manipulating the levels of expression of the positive marker alleles which are sensitive to these levels due to heterochromatic organization or adjacent regulatory elements that may affect the expression of the positive marker or may affect the process of site-specific homologous recombination may be targeted successfully. Table IV lists the more common regulatory regions which may be utilized in the presently described invention.

TABLE IV
Regulatory Regions Used to Drive Selectable Marker Expression

| Regulatory Region | Origin |
|---|---|
| Phosphoglycerate Kinase (PGK) | Mammalian |
| SV-40 (early) | Mammalian, viral |
| Cytomegalovirus (CMV) | Viral |
| Rous sarcoma virus (RSV) | Viral |
| Moloney murine leukemia virus | Viral |
| (MMLV) | |
| MC1 | Viral |

The length of the PPS or cotransformation vector required for successful site-specific homologous recombination is a critical parameter that is often dependent upon the particular gene targeted for creating a mutated DNA sequence. Vector length is dependent upon several factors. The choice of the DNA sequences encoding the positive selectable markers will affect the overall vector length due to the variation of sequence composition for different markers. In addition, in a replacement vector the lengths of DNA sequences one and three, the two sequences which are substantially homologous to cellular endogenous genomic target DNA sequences are crucial parameters that must be correctly addressed for successful gene targeting. In general, one region of homology may be as small as 25 bp (Ayares et al. (1986), Genetics, 83, 5199), although it is recommended that significantly larger regions of homology be utilized. Up to a certain length, an increase in the amount of homology provided in the PPS vector increases targeting efficiency (Zhang et al. (1994), Mol. Cell Biol., 14, 2404). In most cases the entire vector length will be a minimum of 1 kb and usually will not exceed a maximum of 500 kb, although vector length is also dependent upon the technology utilized to construct the vector. It is possible, for example, to construct a PPS vector with a cosmid, BAC, or YAC as the provider of the two regions of substantial homology thus generating a significantly large vector (Ananvoranich et al. (1997), Biotechniques, 23, 812; Cocchia et al., (2000), Nucleic Acids Res., 28, E81). Vector length also includes plasmid backbone sequences such as those encoding the origin of replication and bacterial drug resistance products such as ampicillin if these are not removed prior to transformation of cells with the vector.

PPS and cotransformation vector DNA sequences which are substantially homologous to cellular endogenous genomic DNA sequences and undergo site-specific homologous recombination for the purpose of creating mutated DNA sequences in cellular targets are preferred to have significantly high homology to cellular counterparts. High homology allows for efficient base pairing during the crossover and strand exchange process of site-specific homologous recombination. Any mismatch base pairing between PPS and cellular DNA sequences disfavors the recombination reaction. It is preferable, for example, that DNA sequences one and three in a PPS replacement vector are 100% homologous to cellular endogenous genomic DNA sequences, less preferable that they are 80% homologous and even less preferable that they are 50% homologous. The second and fourth DNA sequences which encode positive selectable markers are generally nonhomologous to cellular endogenous genomic DNA sequences and therefore do not undergo site-specific recombination with these sequences.

In certain cases it may be advantageous to remove DNA sequences encoding positive selectable markers which have been incorporated into the genome of cells upon site-specific homologous recombination between PPS or cotransformation vectors and cellular endogenous genomic target DNA sequences. This is due to the potential negative effects expression of the positive selectable marker may have on cellular or organismal viability and survival. Alternatively, regulatory elements introduced into the genome of the host cell may adversely affect the expression of endogenous loci juxtaposed to these elements. The removal of sequences encoding positive selectable markers and corresponding regulatory elements is possible by a number of methodologies. The Cre-Lox technology may be successfully applied for the removal of specific sequences introduced into cellular endogenous genomic DNA via PPS or cotransformation vectors and technology (for review on Cre-Lox see Ryding et al. (2001), J Endocrinol., 171, 1). For example, sequences encoding a positive selectable marker and corresponding regulatory elements may be flanked with LoxP recombination sites in the PPS vector prior to cellular transformation. After introduction of these sequences into the genome of the host cell a transient or stable expression of the Cre recombinase will allow for removal of one LoxP site and all sequences positioned between the LoxP sites. Many examples of the application of Cre-lox technology for sequence removal exist. Kaartinen et al. have demonstrated removal of a neomycin phosphotransferase cassette flanked by lox P site through the transient expression of Cre via adenoviral infection of 16-cell-stage morulae (Kaartinen et al. (2001), Genesis, 31, 126). Xu et al. successfully removed a lox P flanked neomycin phosphotransferase cassette through both a cross with mice expressing Cre under the control of the Ella promoter as well as pronuclear injection of cells containing the cassette with a Cre-expressing plasmid (Xu et al. (2001), Genesis, 30, 1). Thus, if the PPS or cotransformation vector is configured to replace or correct cellular exonic sequences which are defective, such as may be the case for human gene therapy, the positive selectable marker and corresponding regulatory elements may be removed after completion of site-specific homologous recombination between the PPS or cotransformation vector and host DNA.

The PPS and cotransformation vectors and methodology described herein may also be utilized for the purposes of mutating DNA sequences in plants. Indeed, several examples of homologous recombination in plant lineages exist (Siebert, et al. (2002), Plant Cell, 14, 1121 and for review see Schaefer, D. G. (2002), Annu. Rev Plant Physiol. Plant Mol. Biol., 53, 477). In addition, said homologous recombination has been exploited utilizing positive-negative selection technology to target several plant loci including the alcohol dehydrogenase and protoporphyrinogen oxidase (PPO) loci (Xiaohui et al., (2001), Gene, 272, 249; Hanin et al., (2001), Plant J, 28, 671). It is postulated that there are a number of resistance markers which may be utilized for the purposes of implementing PPS and cotransformation methodology to generate mutated DNA sequences via site-specific homologous recombination. These include neomycin phosphotransferase as well as any herbicide or insecticide resistance loci which may allow for a positive selectable characteristic upon introduction into plant cells. Mutations in plants created utilizing PPS and cotransformation vectors and methodology may encompass loss-of-function, gain-of-function or modifications in the expression levels of endogenous loci through the introduction of exogenous regulatory elements. Loss-of-function or gain-of-function mutations may be generated through the ablation of specific endogenous DNA sequences or the alteration of sequences which may change the amino acid composition encoded by a particular plant gene. In addition, “knockin” experiments may be performed in plants through the use of PPS or cotransformation vectors and methodology to introduce an exogenous gene or coding region into an endogenous locus.

Introduction of the PPS or cotransformation vector into plant cells may be accomplished by a variety of methods including those previously developed for the insertion of exogenous DNA into protoplasts (Hain et al. (1985), Mol. Gen. Genet., 199, 161; Negrutiu et al. (1987), Plant Mol. Bio., 8, 363; Paszkowski et al. (1984), EMBO J., 3, 2717). Microinjection may also allow for the successful introduction of the PPS or cotransformation vector into plant cells (De la Pena et al. (1987), Nature, 325, 274; Crossway et al. (1986), Mol. Gen. Genet., 202, 179). In addition, it is possible to introduce the PPS or cotransformation vector into plant cells via liposome-mediated transfection (Deshayes et al. (1985), EMBO J., 4, 2731). Upon successful introduction of the PPS or cotransformation vector into plant cells site-specific homologous recombination may allow for the mutation of cellular endogenous genomic DNA sequences according to the construction and organization of the PPS or cotransformation vector.

The cell separation strategies described in the present invention include cell sorting through the utilization of a FACStar Plus cell sorter as well as manual separation techniques, but the invention is not limited to this apparatus or to these separation techniques. Other cell sorting apparatuses may also be implemented for the effective separation of cells which express one selectable marker verses another selectable marker or no selectable marker. These include, but are not limited to, the FACS Vantage SE I, and FACS Vantage SE II or any apparatus capable of sorting cells based upon methods described in the present invention.

The PPS vector is used in the PPS method to select for transformed target cells containing the positive selection marker. Such positive-positive selection procedures substantially enrich for those transformed target cells wherein homologous recombination has occurred. As used herein, “substantial enrichment” refers to at least a two-fold enrichment of transformed target cells as compared to the ratio of homologous transformants versus non-homologous transformants, preferably a 10-fold enrichment, more preferably a 1000-fold enrichment, most preferably a 10,000-fold enrichment, i.e., the ratio of transformed target cells to transformed cells. In some instances, the frequency of homologous recombination versus random integration is of the order of 1 in 1000 and in some cases as low as 1 in 10,000 transformed cells. The substantial enrichment obtained by the PPS vectors and methods of the invention often result in cell populations wherein about 1%, and more preferably about 20%, and most preferably about 95% of the resultant cell population contains transformed target cells wherein the PPS vector has been homologously integrated. Such substantially enriched transformed target cell populations may thereafter be used for subsequent genetic manipulation, for cell culture experiments or for the production of transgenic organisms such as transgenic animals or plants.

The following Examples are presented by way of example and is not to be construed as a limitation on the scope of the invention.

### EXAMPLES

### Example 1

### Inactivation of the ptch2 Locus Through the Utilization of PPS Vectors and Methods in ES Cells

1. ptch2 Targeting Vector Construction

ptch2 is a transmembrane domain receptor speculated to play a role in the modulation of hedgehog signaling during embryonic development and postnatally (Motoyama, J. et al. (1998), Nat. Genet., 18, 104; Carpenter, D. et al., PNAS, 95, 13630). The ptch2 targeting vector, termed P2TVG, was constructed from a lambda phage mouse genomic DNA library utilizing a phage clone, termed G8-11, which contained genomic sequences spanning exons 5 through 11, which contain transmembrane domains 2 through 8 of the ptch2 receptor (FIG. 1). Briefly, a 1.7 kb 3′ region of homology was amplified from genomic DNA isolated from the ptch2 phage clone G8-11 by PCR and flanked with Kpn1 and Not1 sites. The fragment was subcloned into the pPolylinker plasmid and the plasmid therein after referred to as pPolylinker 1.7. A 5′ region of homology containing exons 5, 6 and the most 5′ region of exon 7 was removed from the genomic clone with the restriction enzymes BamH1 and Nco1, filled in with Klenow fragment DNA polymerase and blunt subcloned into an Hpa1 site of pPolylinker 1.7 and the plasmid therein referred to as pPolylinker 1.7 upper. A DNA fragment encoding the antibiotic resistance marker neomycin phosphotransferase under the control of the phosphoglycerate kinase (PGK) promoter was inserted between the 5′ and 3′ regions of homology to replace coding regions for transmembrane domains 2, 3 and 4, thus inactivating the receptor, and the plasmid designated P2TV. A DNA fragment encoding green fluorescent protein (GFP) under the control of the cytomegalovirus (CMV) promoter was subcloned upstream of the 5′ region of homology and the vector therein referred to as P2TVG (FIG. 1).

2. Transformation of ES Cells With ptch2 Targeting Vector

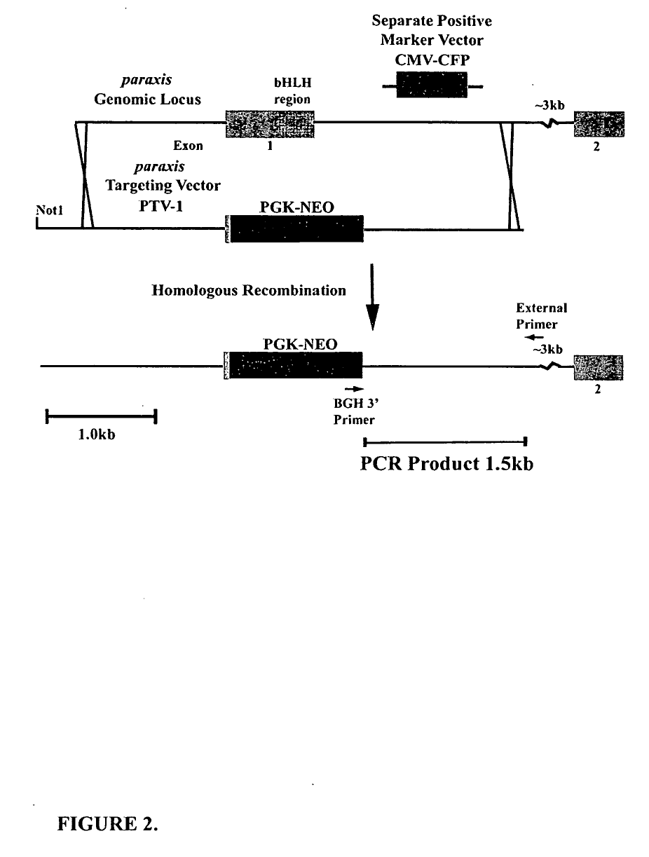

A Not1 site present at the 3′ end of the targeting vector just downstream of the 3′ region of homology was utilized for linearization prior to embryonic stem cell transformation. 100 ug of P2TVG vector was linearized, phenol/chloroform extracted, ethanol precipitated and resuspended in sterile filtered water at a concentration of 1 ug/ul prior to embryonic stem cell transformation. Stem cells were propagated at 37 deg. C, 5% CO₂ on gelatinized 10 cm plates to approximately 50% confluency in M15 media containing 15% FCS, 0.1 mM non-essential amino acids, 1 mM sodium pyruvate, 10⁻⁴ M B-mercaptoethanol, 2 mM L-glutamine, 50 ug/ml penicillin, 50 ug/ml streptomycin, 1000 U/ml LIF in Dulbecco's minimal essential medium (DMEM). Cells were rinsed in media-free DMEM and 8 ug linearized vector per 10 cm plate of ES cells introduced via lipofection techniques with Lipofectamine Reagent according to the manufacturer's specifications (Invitrogen, Inc.). 24-48 hours post transfection cells were either harvested for separation in a FACStar Plus cell sorter or put under antibiotic selection as described below (FIG. 2). Cell harvesting included two rinses in sterile filtered phosphate buffered saline (PBS) followed by trypsinization in 1 ml of 0.05% trypsin/EDTA per 10 cm plate for 15 minutes. Excess trypsin was removed and cells resuspended in cell sorting buffer containing 1 mM EDTA, 25 mM HEPES, pH 7.0 and 1% dialyzed FCS in PBS at a density of 10*10⁶ cells/ml. Cells were kept on ice in 5% CO₂ prior to sorting.

3. Separation of ptch2 Targeted and Nontargeted ES Cells

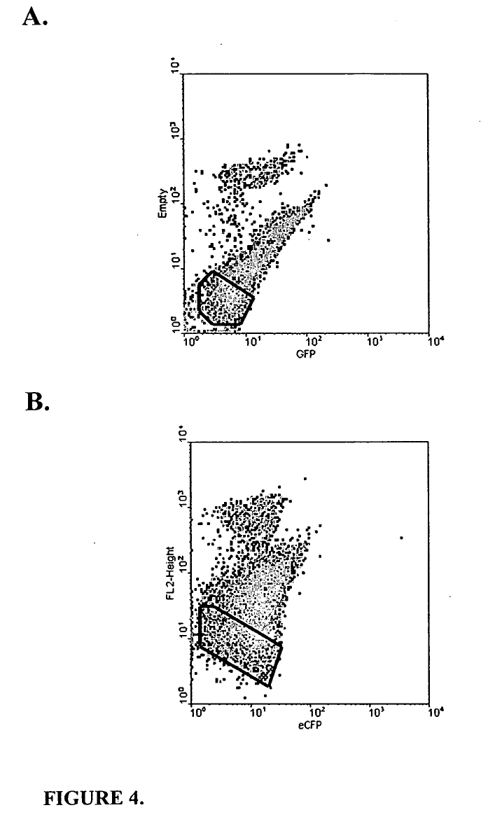

a) ES cells transfected with P2TVG were put under selection in 200 ug/ml geneticin (G418) to select for cells which had incorporated the positive selectable marker neomycin phosphotransferase stably. Cells were selected for 12 days, harvested as described above and bulk sorted in a FACStar Plus cell sorter to separate cells not expressing GFP from those which express it (FIG. 4). Sorted cell populations excluding GFP as well as unsorted cells were replated at a density of 10*10⁶ cells/10 cm plate and propagated to 80% confluency for subsequent isolation of DNA and genotyping.

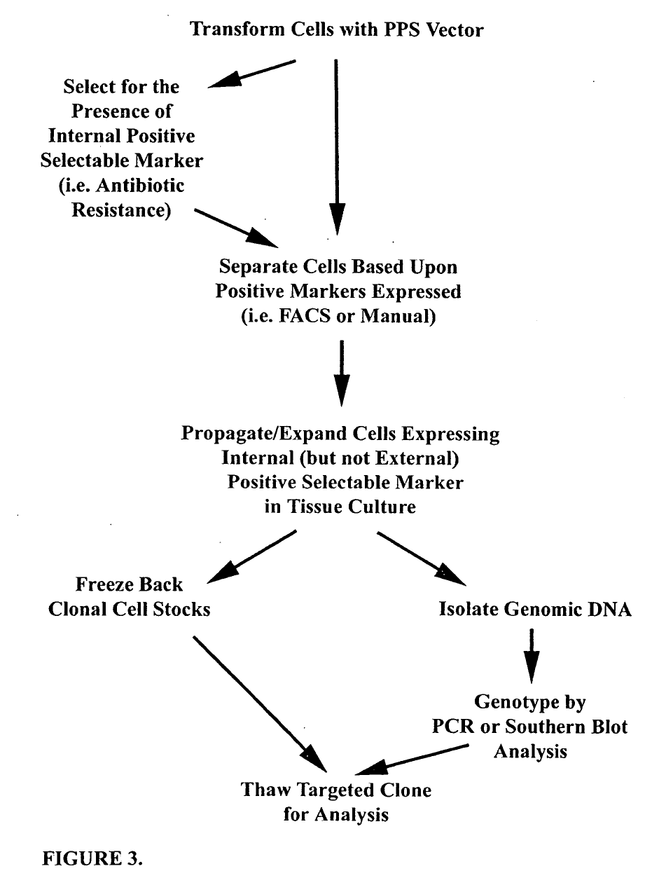

b) Alternatively, ES cells were harvest 48 hours post-transfection without the implementation of antibiotic selection and bulk sorted in a FACStar Plus cell sorter to separate cells not expressing GFP from those which express it (FIG. 3). Sorted cell populations excluding GFP were replated at a density of 10*10⁶ cells/10 cm plate and propagated to 80% confluency for isolation and DNA and genotyping. Said propagation was implemented with cells under selection in 200 ug/ml geneticin (G418) to select for cells which had incorporated the positive selectable marker neomycin phosphotransferase stably. Unsorted cells were propagated to 80% confluency but not subjected to G418 selection.

4. Genotyping Confirmation of ptch2 Mutation by Site-Specific Homologous Recombination

Genomic DNA was isolated from either sorted ES cell populations or unsorted negative control cells by the following protocol. Cells were grown in 10 cm plates to approximately 80% confluence and 1 ml lysis buffer containing 100 mM sodium chloride, 50 mM Tris-HCl, pH 7.5, 10 mM EDTA and 0.5% sodium dodecyl sulfate (SDS) added directly to the plates. Cells were incubated for 15 minutes at room temperature, transferred to 1.5 ml Eppendorph tubes and incubated at 55 deg. C overnight with gentle shaking. Lysates were extracted two times with an equal volume of 1:1 phenol/chloroform and one time with chloroform. Genomic DNA was precipitated with an equal volume of isopropanol. After centrifugation at 15000XG genomic DNA pellets were resuspended in 300 ul sterile filtered water.

Genomic DNA from each sample was genotyped by PCR utilizing an oligonucleotide primer specific for sequences in the PGK promoter and an oligonucleotide specific for sequences just downstream of the 3′ region of homology (FIG. 1). 20 pmoles of each oligonucleotide were mixed with 100 ng genomic DNA in the presence of 200 uM final concentration of each dNTP, 2.5 mM MgCl₂, 1×PCR buffer and 1 U Taq DNA polymerase (Invitrogen, Inc.). Amplification was performed through application of the following cycling parameters: 94.0 deg. C for 2 minutes followed by 35 cycles of 96 deg. C for 30 seconds, 58 deg. C for 30 seconds and 72 deg. C for 2.5 minutes. Reactions were electrophoresed in parallel with Ikb ladder molecular weight standards on a 0.8% agarose gel and the gel stained with ethidium bromide for UV detection of PCR products. A 10.7 kb PCR product was detected utilizing DNA from sample populations sorted to exclude GFP for both cells which had been selected in G418 prior to as well as post-sorting indicating site-specific homologous recombination and successful gene targeting. No product was observed utilizing DNA from unsorted cells or in negative controls.

### Example 2

### Inactivation of the ptch2 Locus Through the Utilization of Cotransformation Methods in ES Cells

1. Cotransformation of ES Cells With ptch2 Targeting and CFP

The cotransformation methodology relies on the fact that two types of integration may occur upon transformation. Type I is site-specific homologous recombination which introduces a single integrant into the genome of the cell. Type II is random concatemeric integration which introduces vector multimers (FIG. 6). Embryonic stem cells grown to approximately 50% confluency were cotransfected with 4 ug each of linearized P2TV (no fluorescent protein cassette) and a separate linearized vector encoding cyan fluorescent protein (CFP) under the control of the CMV promoter. Cotransfections were accomplished via lipofection protocols as described above according to manufacturer's specifications (Invitrogen, Inc.). Cells were harvested 48 hours post-transfection and resuspended in cell sorting buffer as described above.

2. Separation of ptch2 Targeted and Nontargeted ES Cells

Cells were bulk sorted in a FACStar Plus cell sorter to separate cells not expressing CFP from those which express it (FIG. 4). Sorted cell populations were replated at a density of 10*10⁶ cells/10 cm plate and propagated to 80% confluency for isolation and DNA and genotyping. Said propagation was implemented with cells under selection in 200 ug/ml geneticin (G418) to select for cells which had incorporated the positive selectable marker neomycin phosphotransferase stably. Unsorted cells were propagated to 80% confluency but not subjected to G418 selection.

3. Genotyping Confirmation of ptch2 Mutation by Site-Specific Homologous Recombination

Genomic DNA was isolated from either sorted ES cell populations or unsorted negative control cells as described above. Genomic DNA from each sample was genotyped by PCR utilizing an oligonucleotide primer specific for sequences in the PGK promoter and an oligonucleotide specific for sequences just downstream of the 3′ region of homology (FIG. 1). Reaction volumes and conditions are as described above. Reactions were electrophoresed in parallel with 1 kb ladder molecular weight standards on a 0.8% agarose gel and the gel stained with ethidium bromide for UV detection of PCR products. A 10.7 kb PCR product was detected utilizing DNA from sample populations sorted to exclude CFP indicating site-specific homologous recombination and successful gene targeting, but no product was observed utilizing DNA from unsorted cells or in negative controls.

### Example 3

### Inactivation of the ptch2 Locus Through the Utilization of Cotransformation Methods in 3T3 Cells

1. Cotransformation of 3T3 Cells With ptch2 Targeting Vector and GFP

NIH 3T3 fibroblasts were cultured in 10% FCS in DMEM containing 1 mg/ml ciprofloxacin on 10 cm plates to approximately 50% confluency and cotransfected with 4 ug each of linearized P2TV (no fluorescent protein cassette) and a separate linear vector encoding green fluorescent protein (GFP) under the control of the CMV promoter. Cotransfections were accomplished via lipofection protocols as described above according to manufacturer's specifications (Invitrogen, Inc.). 24 hours post transfection cells were selected in 11.0 mg/ml G418 for 14 days to allow for the selected survival and manual isolation of clonal colonies expressing the neo resistance marker.

2. Manual Separation of ptch2 Targeted and Nontargeted 3 T3 Cells

48 colonies which had survived G418 selection were manually picked, trypsinized in the presence of 50 ul 0.05% trypsin and replated in 35 mm plates (FIG. 3). Colonies were subsequently grown to confluency in the presence of 11.0 mg/ml G418 and observed for emission of fluorescence at wavelengths consistent with GFP. 13 of the 48 colonies emitted no observable fluorescence (see Table V). These clones were pursued further to determine whether or not site-specific homologous recombination had occur at the ptch2 locus between endogenous genomic DNA sequences and the P2TV vector.

3. Genotyping Confirmation of ptch2 Mutation by Site-Specific Homologous Recombination

Cells were grown to confluency, lysed and genomic DNA was isolated from each clonal line as described above. Genomic DNA from each sample was genotyped by PCR utilizing an oligonucleotide primer specific for sequences in the PGK promoter and an oligonucleotide specific for sequences just downstream of the 3′ region of homology (FIG. 1). Reaction volumes and conditions are as described above. Reactions were electrophoresed in parallel with Ikb ladder molecular weight standards on a 0.8% agarose gel and the gel stained with ethidium bromide for UV detection of PCR products. A 1.7 kb PCR product was detected in 2 out of the 13 lines indicating site-specific homologous recombination and successful gene targeting and a targeting efficiency of approximately 15.3% (see Table V). No product was observed utilizing DNA from negative controls.

TABLE V

| | |
|---|---|
| Number of Colonies Isolated | 48 |
| Number of Isolated Colonies not | 13 |
| Fluorescing | |
| Number of Nonfluorescing Colonies | 2 |
| Undergoing Homologous Recombination | |
| Targeting Efficiency | 15.3% |

### Example 4

### Inactivation of the paraxis Locus Through the Utilization of Cotransformation Methods in ES Cells

1. paraxis Targeting Vector Construction

paraxis is a basic helix-loop-helix transcription factor implicated in the control of somite formation during mammalian embryogenesis (Burgess, R. et al., (1995), 168, 296; Burgess, R. et al., (1996), Nature, 384, 570; Barnes, G. L. et al. (1997), Dev. Biol., 189, 95). The construction of the paraxis targeting vector has been previously described (Burgess, R. et al., (1996), Nature, 384, 570). The paraxis genomic organization consists of two exons separated by a 5 kb intron. The first exon contains the initiating methionine codon and the basic helix-loop-helix (bHLH) domain responsible for DNA binding and dimerization. The first exon was chosen for deletion to remove sequences including the initiating methionine through the bHLH domain, thus inactivating the paraxis protein product. Neomycin phosphotransferase under the control of the PGK promoter was utilized to replace the majority of exon 1 as well as 5′ regions of intron 1 (FIG. 2). Note: The HSV-thymidine kinase cassette was removed from the existing PTV-1 vector and thus no selection for the presence of this marker was implemented.

2. Cotransformation of ES Cells With a paraxis Targeting Vector and CFP

Embryonic stem cells grown to were grown to approximately 50% confluency and cotransfected with 4 ug each of linearized PTV-1 (no fluorescent protein cassette) and a separate linear vector encoding cyan fluorescent protein (CFP) under the control of the CMV promoter. Cotransfections were accomplished via lipofection protocols as described above according to manufacturer's specifications (Invitrogen, Inc.). 48 hours post-transfection cells were harvested without the implementation of antibiotic selection as described above and bulk sorted in a FACStar Plus cell sorter to separate cells not expressing CFP from those which express it. Sorted cell populations excluding CFP were replated at a density of 10*10⁶ cells/10 cm plate and propagated to 80% confluency for isolation and DNA and genotyping. Said propagation was implemented with cells under selection in 200 ug/ml geneticin (G418) to select for cells which had incorporated the positive selectable marker neomycin phosphotransferase stably. Unsorted cells were propagated to 80% confluency but not subjected to G418 selection. 3.

4. Genotyping Confirmation of paraxis Mutation by Site-Specific Homologous Recombination

Genomic DNA was isolated from either sorted ES cell populations or unsorted negative control cells as described above. Genomic DNA from each sample was genotyped by PCR utilizing an oligonucleotide primer specific for sequences in the bovine growth hormone polyadenylation signal 3′ of neo coding sequences and an oligonucleotide specific for sequences just downstream of the 3′ region of homology (FIG. 2). Reaction volumes and conditions are as described above with the exception of the primer annealing temperature which was 55 deg. C. Reactions were electrophoresed in parallel with Ikb ladder molecular weight standards on a 0.8% agarose gel and the gel stained with ethidium bromide for UV detection of PCR products. A 1.5 kb PCR product was detected utilizing DNA from sample populations sorted to exclude CFP indicating site-specific homologous recombination and successful gene targeting, but no product was observed utilizing DNA from unsorted cells or in negative controls.

Having described the preferred embodiments of the present invention, it will appear to those ordinarily skilled in the art that various modifications may be made to the disclosed embodiments, and that such modifications are intended to be within the scope of the present invention. Although the invention has been described with reference to the above examples, it will be understood that modifications and variations are encompassed within the spirit and scope of the invention. Accordingly, the invention is limited only by the following claims.

1. A method for identifying a transformed cell which has undergone site-specific homologous recombination utilizing a PPS vector comprising:
a) transforming cells with a PPS vector designed to undergo site-specific homologous recombination wherein the vector comprises:
a first DNA sequence which is substantially homologous to an endogenous genomic sequence present within the host genome;
a second DNA sequence which encodes a positive selection characteristic in said cells and is non-homologous to cellular endogenous genomic sequences and therefore incapable of undergoing site-specific homologous recombination;
a third DNA sequence which is substantially homologous to an endogenous genomic sequence present within the host genome and is different from the first DNA sequence; and
a fourth DNA sequence which encodes a positive selection characteristic in said cells and is non-homologous to a cellular endogenous genomic sequence and therefore incapable of undergoing site-specific homologous recombination;
b) propagating cells to select for or enrich for those which have been transformed with said PPS vector by selecting for the presence of the positive selectable marker gene product of said second DNA sequence; and
c) separating cells which have said second DNA sequence encoding a positive selectable marker from cells which have said fourth DNA sequence encoding a positive selectable marker.

2. The method of claim 1, further comprising
d) characterizing the genomic DNA of said cells carrying the second DNA sequence encoding a positive selectable marker but not carrying the fourth DNA sequence encoding a positive selectable marker for the site-specific homologous recombination events which allow for modification of the cellular target DNA.

3. The method of claim 1 wherein said PPS vector includes positive selectable markers detectable by addition of antibiotics to cell cultures.

4. The method of claim 1 wherein said PPS vector includes positive selectable markers which may be detected by fluorescence light emission.

5. The method of claim 1 wherein said positive selectable markers allow for the separation of cells containing DNA encoding one marker from cells containing DNA encoding another or both markers.

6. The method of claim 1 wherein said cells are capable of homologous recombination.

7. The method of claim 1 wherein said cells are from a multicellular organism.

8. (Cancelled).

9. The method of claim 1 wherein said cells have undergone multiple rounds of site-specific homologous recombination for the purposes of multiple modifications of the endogenous cellular genome.

10. The method of claim 1 wherein said cells may be utilized to create a multicellular organism.

11. The method of claim 1 wherein said cells are embryonic stem cells.

12. An isolated PPS vector for site-specific homologous recombination in cells capable of undergoing homologous recombination, the vector comprising:
a first DNA sequence which is substantially homologous to cellular endogenous genomic sequences and is capable of undergoing homologous recombination in said cells,
a second DNA sequence which is nonhomologous to cellular endogenous genomic sequences, is not capable of undergoing homologous recombination in said cells, and encodes a positive selectable marker capable of allowing for the identification of cells containing said positive selectable marker,
a third DNA sequence which is substantially homologous to cellular endogenous genomic sequences and is capable of undergoing homologous recombination in said cells,
a fourth DNA sequence which is nonhomologous to cellular endogenous genomic sequences, is not capable of undergoing homologous recombination in said cells, and encodes a positive selectable marker which allows for the separation of cells containing said positive selectable marker from cells not containing said positive selectable marker
wherein the organization of said PPS vector in 5′ to 3′ orientation comprises: the first DNA sequence which is substantially homologous to cellular endogenous genomic DNA sequences, the second DNA sequence which encodes a positive selectable marker, the third DNA sequence which is substantially homologous to cellular endogenous genomic DNA sequences, and the fourth DNA sequence which encodes a positive selectable marker;
wherein the vector is capable of undergoing site-specific homologous recombination resulting in modification of cellular endogenous target genomic DNA sequences.

13. The PPS vector of claim 12 wherein said cellular endogenous genomic target DNA is comprised of exons and introns.

14. The PPS vector of claim 13 wherein said vector contains all or portions of exons and introns which are substantially homologous to cellular target genomic DNA sequences.

15. The PPS vector of claim 12 wherein said vector contains all or portions of regulatory elements which are substantially homologous to cellular target genomic DNA sequences.

16. The PPS vector of claim 12 wherein said vector contains alterations in sequences which are substantially homologous to cellular target genomic DNA sequences including deletions, substitutions, additions or point mutations.

17. The PPS vector of claim 12 wherein said positive selection marker encoded by said fourth DNA sequence is selected from DNA sequences encoding fluorescent proteins including GFP, CFP, YFP, RFP, dsRED or HcRED.

18. The PPS vector of claim 12 wherein said positive selection marker encoded by said second DNA sequence is selected from a group of DNA sequences encoding resistance markers including neo, puro, blasticidin, bleomycin, zeocin or hygro.

19. The PPS vector of claim 12 wherein said positive selection marker encoded by said second DNA sequence is selected from a group of DNA sequences encoding fluorescent proteins including GFP, CFP, YFP, RFP, dsRED and HcRED.

20. The PPS vector of claim 12 wherein said vector may include an additional fifth DNA sequence which is nonhomologous to cellular endogenous genomic DNA sequences, is positioned external to said first and third DNA sequences on the opposite side containing said fourth DNA sequence which encodes a positive selectable marker and encodes a positive selectable marker.

21. The PPS vector of claim 12 wherein said fourth and fifth DNA sequences may encode positive selectable markers which allow for the separation of cells containing DNA encoding said selectable markers from cells which do not contain DNA encoding said selectable markers.

22. The PPS vector of claim 12 wherein said vector has lengths of homology for said first and third DNA sequences which are between about 50 bp and 50,000 base pairs.

23. The PPS vector of claim 12 wherein said vector results in the modification of cellular endogenous genomic target DNA sequences.

24. The PPS vector of claim 12 wherein said vector introduces exogenous regulatory elements into the cellular endogenous genomic target DNA sequences.

25. An enriched population of cells generated through a method according to claim 1 wherein said cells have undergone site-specific homologous recombination.

26. A non-human transgenic animal generated by the method of claim 1 wherein said animal has been generated from cells which have undergone site-specific homologous recombination.

27-51. (Cancelled).
